(19) United States
(12) Patent Application Publication
(10) Pub. No.: US 2022/0296928 A1
(43) Pub. Date: Sep. 22, 2022

(54) LASER SKIN MARKING FOR RADIATION THERAPY (RT) PLANNING

(71) Applicant: KONINKLIJKE PHILIPS N.V., EINDHOVEN (NL)

(72) Inventor: Steffen WEISS, HAMBURG (DE)

(21) Appl. No.: 17/619,779

(22) PCT Filed: Jun. 10, 2020
(86) PCT No.: PCT/EP2020/066016

§ 371 Date: Dec. 16, 2021

Related U.S. Application Data
(60) Provisional application No. 62/862,974, filed on Jun. 18, 2019.

(51) Int. Cl. A61N 5/10

(52) U.S. Cl. CPC A61N 5/1049; A61B 2090/395

(57) ABSTRACT

A positioning device (12) for use in RT includes one or more dye marker light sources (32) disposed in fixed position with respect to the medical device and configured to emit activating light onto the patient to be imaged or treated with the medical device which is effective to visually mark an associated photochromic dye (34) disposed on the patient.

### FIELD

The following relates generally to RT arts, patient positioning arts, RT location marking arts, medical imaging arts, and related arts.

### BACKGROUND

Current RT systems use magnetic resonance imaging (MRI) devices, computed tomography (CT) imaging devices, a hybrid MRI/CT system, or the like, to acquire planning images of tumor and risk organs (e.g., healthy organs that should receive a minimal RT dose). Based on these images, a dose plan is prescribed which provides the basis of the calculation of the beam geometry, beam shape, irradiation times, and/or other parameters of a radiation therapy system employed during delivery of external beam RT.

This workflow requires the registration of the co-ordinate systems of the imaging systems with that of the therapy system. To do so, the patient is positioned on a support used to load the patient into the imaging system. The patient is then marked with (typically) three points (e.g., one anterior mark and two lateral marks) on the patient/s skin corresponding to the (e.g.) three points of a light pattern projected onto the skin of the patient using a first laser bridge that is located near the imaging system and that has a fixed position respective to the frame of reference of the imaging system. The acquired images are then used to develop the dose plan. Sometime later (e.g., days later or longer in many cases) the patient receives radiation therapy according to the dose plan. The patient is positioned on a support used to load the patient into the therapy system. This support typically has identical shape and size as the support used with the imaging system. Then, the position of the patient is adjusted to align the skin markings with a (e.g. three point) light pattern projected onto the skin of the patient using a second laser bridge that is located near the therapy system and that has a fixed position respective to the frame of reference of the therapy system. In this way, it is assured that the patient's position in the frame of reference of the therapy system matches the patient's position in the frame of reference of the imaging system.

The markings are typically applied to the patient's skin during the planning imaging phase by applying tattoos by way of injection of ink with a syringe, or by manual marking of the points using a pen, or by placing adhesive markers onto the patient's skin (see, e.g., Rathod S, Munshi A, Agarwal J. Skin markings methods and guidelines: A reality in image guidance radiotherapy era. South Asian Journal of Cancer. 2012; 1(1):27-29. doi:10.4103/2278-330X.96502). The use of three points for marking the trunk of the patient ensures full spatial alignment with spatial inaccuracies of typically 2 mm translation and few degrees in a yaw axis (see, e.g., Elsner K, Francis K, Hruby G, Roderick S. Quality improvement process to assess tattoo alignment, set-up accuracy and isocentre reproducibility in pelvic radiotherapy patients. J Med Radiat Sci. 2014 December; 61(4):246-252).

The following discloses new and improved systems and methods to overcome these problems.

### SUMMARY

In one disclosed aspect, a positioning device for use in RT includes one or more dye marker light sources disposed in fixed position with respect to the medical device and configured to emit activating light onto the patient to be imaged or treated with the medical device which is effective to visually mark an associated photochromic dye disposed on the patient.

In another disclosed aspect, a medical system includes one or more visible light sources disposed on an outer circumference of a bore of a medical imaging device and configured to emit a visible light pattern comprising one or more lines or shapes onto a patient to be imaged or treated with the medical imaging device. One or more marker light sources are disposed on an outer circumference of the bore and configured to emit activating light onto the patient to be imaged or treated with the medical imaging device which is effective to visually mark skin of the patient or an associated photochromic dye disposed on skin of the patient with a marking comprising one or more lines or shapes coinciding with the one or more lines or shapes of the visible light pattern.

In another disclosed aspect, a method of treating a patient with RT includes: emitting light towards the patient to generate a visible light pattern on skin of the patient overlaying one or more areas to be treated with RT; generating, with a photochromatic dye delivered by one or more dye marker light sources, a marking on the skin of the patient where the visible light pattern is located; and delivering RT to the patient at the area underlying the marking on the skin of the patient.

One advantage resides in eliminating manual skin markings in RT planning to save procedure costs and time.

Another advantage resides in reducing spatial inaccuracies of manual skin markings for RT planning.

Another advantage resides in providing efficient application of more complex skin markings that can facilitate more accurate patient alignment.

Another advantage resides in reducing indentations and dislocations of skin during marking generation for RT planning.

Another advantage resides in providing fast automated skin marking without potentially pain-inducing damage to patient skin.

Another advantage resides in providing automatically generated skin markings with high visual contrast and/or a distinctive color.

Another advantage resides in providing automatically generated skin markings that are easily removed after serving as alignment references during the therapy phase.

Another advantage resides in providing fast skin marking so as to reduce or eliminate the likelihood of patient movement during the skin marking process.

Another advantage resides in enabling enable skin marking in a bore of a medical imaging device to avoid any patient motion between an imaging procedure performed inside the bore and a marking procedure performed outside of the bore.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically an embodiment of a medical system for RT according to one aspect.

FIG. 2 illustrates some contemplated dye marking patterns.

FIG. 3 diagrammatically another embodiment of a medical system for RT according to one aspect.

FIG. 4 shows exemplary flow chart operations of the system of FIG. 1.

### DETAILED DESCRIPTION

The following relates to RT planning. As part of RT planning, medical images are acquired of the anatomical region to be irradiated. The RT plan is designed, based on the planning images, to satisfy dose objectives usually defining some minimum dosage to the tumor and some constraints or limits on the dosage to so-called critical organs, i.e. organs that could be damaged by excessive radiation exposure. The RT is performed after the planning imaging, e.g. days later (and, in fractional RT, in multiple sessions on multiple days).

To accurately irradiate in accordance with the plan, the patient must be aligned in the RT device in the same way as in the imaging device. Conventionally, this is achieved by imaging and RT laser bridges positioned next to the imaging device and the RT device, respectively, to project light markers at a predetermined position respective to the respective frames of reference of the imaging and RT systems. During the planning, the clinician uses ink, tattooing, or an adhesive marker to mark the location of the light marker on the patient's skin (this light marker is projected by the imaging light bridge aligned with the imaging system), and when being loaded into the radiation therapy device the patient is positioned so that ink spot, tattoo, or marker is aligned with the corresponding light marker generated by the RT laser bridge that is aligned with the RT device.

In some embodiments disclosed herein, the imaging light bridge is modified by adding a marking laser to the imaging light bridge of the imaging device which directly marks the patient's skin, or which marks photochromic dye administered to the patient's skin. These approaches improve accuracy as compared with manual marking using a pen, tattooing, or adhesive, since the clinician does not need to touch the patient's skin, and can provide for instantaneous marking of more complex patterns such as lines or shapes that can provide more useful positioning assistance compared with small discrete pen marks, tattoos, or markers. The marking laser is illustrated herein as separate from the light source generating the visually perceptible pattern, although it is alternatively contemplated to use the same light source for both projecting the pattern and marking the skin or dye (e.g. by operating at higher optical power to mark). Embodiments that employ a photochromatic dye have significant advantages over embodiments that directly mark the patient's skin, including being less painful for the patient, permitting the mark to be washed off using a special solution after the radiation therapy (versus direct marking in which the mark constitutes an area of skin damage that remains unless/until the skin heals), using lower laser power versus direct skin marking (in turn reducing or eliminating the need for laser eye protection to be worn by the patient and/or imaging system operators), and providing greater flexibility for aspects such as the color of the laser-formed mark.

The disclosed imaging light bridge embodiments can be used in conjunction with any imaging modality used to acquire planning images for RT planning, and likewise for any RT modality. The marking laser can be an ultraviolet (UV) laser so that the photochromatic dye can be made insensitive to visible light. A marking laser is preferred, but since lower power is needed to mark a photochromatic dye versus direct skin marking some LED/lensing arrangement is also contemplated for the dye marking, and could be useful for generating more complex mark patterns such as lines or shapes. In addition, some complicated shapes (e.g., stars, diamonds, pentagons, hexagons, and the like) can also be produced with laser beams by circulating the laser spot so quickly along the desired shape outline that it is seen as a constant shape. In a contemplated variant for magnetic resonance imaging/linear accelerator (MRI/LINAC) systems, the photochromatic dye could include metallic particles providing visibility of the laser-generated mark in the MRI planning images.

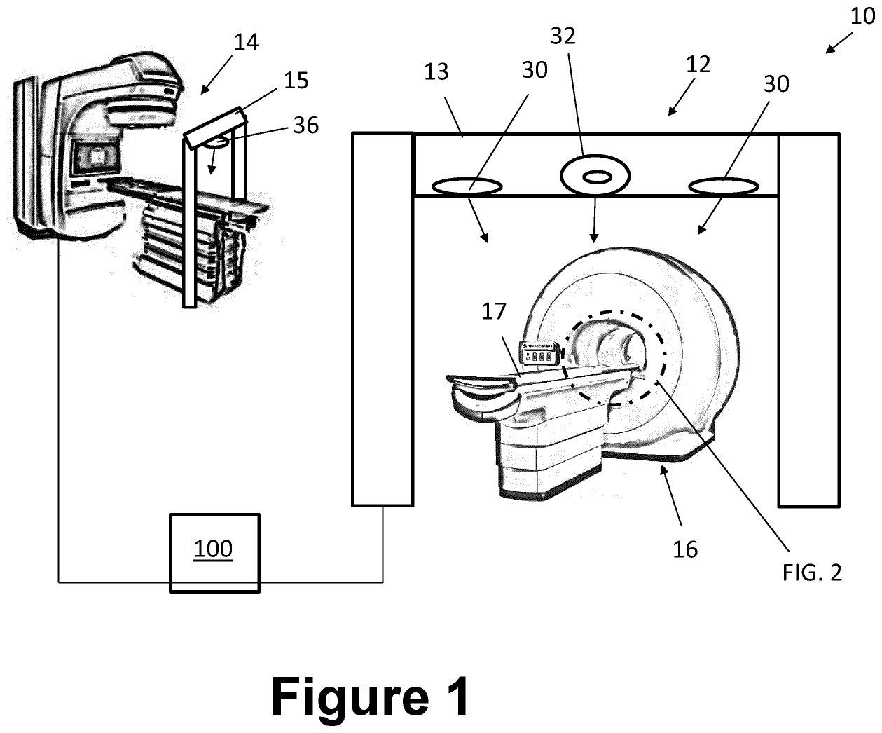

With reference to FIG. 1, an illustrative embodiment of a medical system 10 is shown. The medical system includes a positioning device (e.g., an illustrative imaging light bridge) 12 associated with a medical imaging device 16, a second positioning device (illustrative RT light bridge) 15 associated with a RT device 14, and the medical imaging device 16. Although described here in as an MRI device, the medical imaging device 16 can be any suitable imaging device (e.g., computed tomography (CT); positron emission tomography (PET), single photon emission computed tomography (SPECT), x-ray, ultrasound, or hybrid systems such as a PET/CT device).

The RT device 14 can be any type of RT device employing therapeutic radiation beams, e.g. electron beams, proton beams, high energy X-ray beams, or so forth. The RT may employ a discrete “step-and-shoot” approach in which a radiation beam source is stepped between successive fixed positions along a trajectory that partially or entirely encircles the patient. Alternatively, the RT may employ a continuous arc radiation therapy, such as VMAT, Intensity Modulated Arc Therapy (IMAT), step and short RT delivery, or so forth, in which the radiation beam source continuously irradiates the patient as the beam is revolved around the patient along a partially or entirely encircling trajectory. For example, the traversing of the trajectory comprises moving a therapeutic radiation source along a continuous arc. The number of beams may be one, two, three, or more. In the case of continuous arc radiation therapy, the number of arcs executed in the therapy session may, in general, be one, two, three, or more. For planning purposes, a continuous arc is discretized into discrete control points, e.g. at 2° to 4° intervals (intervals larger or smaller than this are also contemplated, depending upon the desired resolution). In other examples, the trajectory may be circular, non-circular, or otherwise shaped, and may be traversed in a step and shoot approach or continuously. In the latter case, the continuous trajectory is typically discretized into control points along the trajectory in order to make the radiation therapy planning more tractable. In one example, the radiation delivery planning optimization system can be a linear accelerator (LINAC) with a multi-leaf collimator (MLC) configured to shape and deliver a high energy electron beam that strikes a target (e.g., an x-ray or gamma ray generator and associated hardware which serves as a radiation source) that emits x-rays (i.e., photons) in response, resulting in a therapeutic beam delivered to a patient (not shown). These are merely non-limiting illustrative examples.

FIG. 1 also shows the positioning device 12, which is used in the RT planning. The positioning device 12 can be configured as a light bridge 13 positioned as surrounding the patient as the patient lies on a (typically motorized) patient support 17 of the medical imaging device 16. The positioning device 12 includes one or more visible light sources 30 that are disposed in fixed position with respect to the medical imaging device 16 and/or the RT device 14. As shown in FIG. 1, the positioning device 12 includes two visible light sources 30 (although any suitable number of visible light sources can be used). The visible light sources 30 are configured to emit (e.g. project) a visible light pattern onto a patient to be imaged with the medical imaging device 16.

The positioning device 12 at the imaging device 16 also includes one or more dye marker light sources 32 that are disposed in fixed position with respect to the medical imaging device 16. As shown in FIG. 1, the positioning device 12 includes dye marker light source 32 (although any suitable number of dye marker light sources can be used). The dye marker light sources 32 are configured to emit activating light onto the patient to be imaged or treated with the medical device which is effective to visually mark a photochromic dye disposed on the skin of the patient. The mark on the photochromatic dye should be visibly perceptible, and can be a darkening or lightening of the dye, a color-changing mark of the dye, or some other visually perceptible change to the dye disposed on the patient such as a darkening or lightening of a photochromic dye. Preferably, the activating light emitted by the dye marker light sources 32 is not effective to visually mark skin of the patient so that the marking process does not generate skin damage. Furthermore, the mark is not permanent and, depending upon the composition of the photochromatic dye, takes a short time to wash off or otherwise remove. (Typically, the layer of photochromatic dye is removed from the skin by a suitable solvent, repeated washing, or the like, and the mark is thereby removed along with the applied dye). In one example, the emitted activating light induces a color or opacity in the portion of the photochromic dye disposed on the patient that is illuminated by the activating light. In another example, the emitted activating light induces a color change in the portion of the photochromic dye disposed on the patient that is illuminated by the activating light. Preferably, the dye is transparent or translucent prior to exposure onto the patient's skin.

Referring back to FIG. 1, in some embodiments, the pattern produced by the visible light sources 30 is a three-dot pattern such as that conventionally used when marking via a pen, tattoo, or adhesive marker, and the dye marking light source 32 marks the photochromatic dye with a corresponding three-dot pattern.

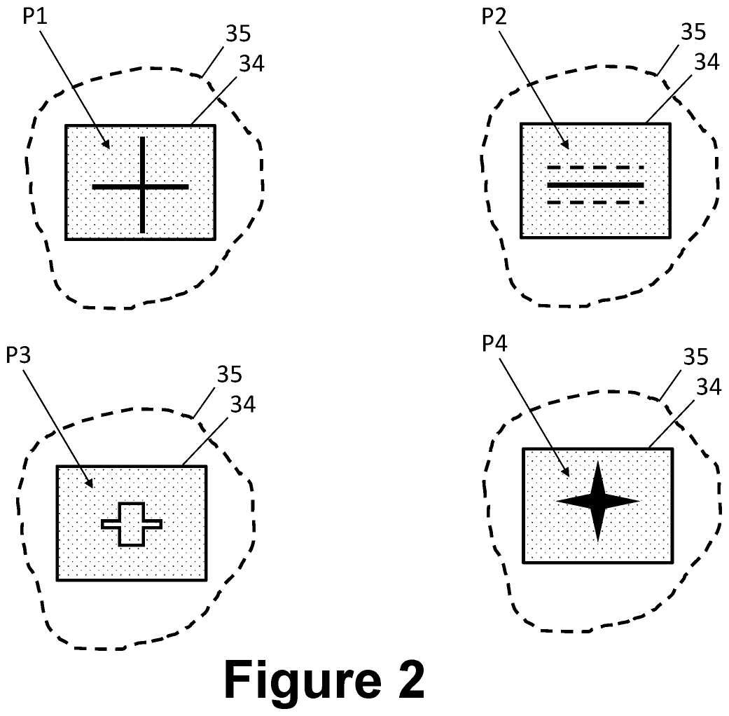

With continuing reference to FIG. 1 and with further reference to FIG. 2, due to the rapid automatic marking process performed by the marking light source 32, along with the observation that the dye marking process does not create skin damage, it is feasible to employ more complex markings, such as marking the dye on the skin with one or more lines. In some examples shown in FIG. 2, a photochromatic dye 34 disposed on skin 35 of the patient is marked by the marker light source(s) 32 with a set of perpendicular lines (e.g., a cross as in illustrative example mark P1 of FIG. 2) or parallel lines (illustrative example mark P2 of FIG. 2) can be used to mark the patient's skin, optionally such that the lines are aligned with an anatomical direction of the patient (e.g. the lines of P2 may be aligned with the craniocaudal axis, and the crossing line of P1 may be aligned with the left-right anatomical axis). The line(s) can have an aspect ratio of at least 3:1, and more preferably 5:1 or higher. The dye 34 can also visually comprise a marking having a longest dimension (e.g., at least three inches). In other examples, the dye 34 can be used to create a shape (e.g., circles, triangles, squares, diamonds, and so forth). This is illustrated in FIG. 2 as an illustrative example hollow cross pattern P3, and a (different) illustrative example solid cross pattern P3. A solid shape such as that of mark P4 can be advantageous as it may have stronger contrast than a hollow shape such as that of mark P3—and yet, since the marking light source is marking a photochromatic dye, the solid shape of mark P4 may be imprinted on the dye 34 on the skin 35 as fast as the hollow mark P3. A marked line can be a continuous line, or can be a dotted or dashed line or other type of broken line (as in the outer broken lines of the example P2). Likewise, a marked shape can have solid and/or broken lines, or the shape can be a solid shape as in the example P4 of FIG. 2. In FIG. 2, the photochromatic dye 34 is coated over a rectangular area of the skin 35. However, other coated area shapes are contemplated, and in some implementations the photochromatic dye may be painted onto the skin manually in which case the skin area coated with the photochromatic dye may be irregular. (Such manual application of the dye is practical since little or no precision is required, the dye merely must cover a sufficient area to be sure to encompass the marking applied by the dye marking light source 32).

As mentioned previously, applying spatially extended marks such as those of illustrative FIG. 2 can be done using embodiments employing the photochromatic dye 34 without introducing skin damage. In alternative embodiments in which the marking light source directly marks the skin, the application of the mark introduces skin damage (which is the mark), and hence it is likely that small marks (e.g. a three-point pattern) will be preferred when using direct skin marking. However, it is contemplated to employ extended marks such as those of FIG. 2 in conjunction with direct skin marking using an optical marking light source, especially if the marking light source is tuned in power and wavelength to limit skin damage to the upper surface of the skin (e.g. by using relatively short, e.g. ultraviolet, wavelength light that has a short penetration depth into the skin).

In some embodiments, the dye marker light sources 32 are effective to visually mark a napthopyran photochromic dye 34 disposed on the patient. A napthopyran photochromic dye is advantageous for several reasons, including: (i) it can change from transparent to colored due to illumination; (ii) such a color change can be induced by UV light; (iii) it can be provided as a liquid solution or a topical cream and applied safely on human skin; (iv) it can be easily be absorbed into skin when dissolved in a formulation containing ethyl alcohol and dimethyl isosorbide which penetrate the skin; (v) it meets U.S. FDA requirements; and (vi) it can block ambient light. However, more generally any photochromatic dye can be used that is biocompatible for coating onto the skin, can be effectively marked by the chosen dye marker light source 32, and is resistant to being washed away during bathing or showering.

In one example embodiment, a user can control operation of the visible light sources 30 and/or the dye marker light sources 32 using light switches (not shown) mounted on the light bridge 13 or elsewhere (e.g. on a workstation of the imaging device 16). In a guidance mode, the one or more visible light sources 30 are turned on using an on/off switch so as to emit the visible light pattern onto the patient, but the marking light source(s) 32 are off in the guidance mode. The patient is then positioned using motorized control of the patient support 17 with the projected light pattern emitted from the visible light sources 30 located at the anatomical location where the operator wants to make the mark (e.g., positioned with the projected light pattern illuminating the pre-applied photochromatic dye 34). To apply the mark, the user suitably presses a “mark” (or similarly labeled) button on the light bridge 17 to turn on the marking light source(s) 32 in order to emit the activating light onto the patient for an exposure time effective to visually mark an photochromic dye 34 disposed on the patient, after which time the marking light source(s) 32 turn off. Typically, a fixed exposure time is employed (i.e. pressing the “mark” button turns on the marking light source(s) 32 for a predetermined fixed time). To accommodate the use of different photochromatic dyes with different exposure times, an exposure time setting control may be provided on the light bridge 13 or elsewhere which allows the operator to set the exposure time.

In another example embodiment, the one or more dye marking light sources 32 comprise the one or more visible light sources 30 and are further configured to operate in a marking mode to emit the activating light which is effective to visually mark the photochromic dye 34 disposed on the patient. For example, the marking mode could be a higher intensity of light, or a shorter wavelength of light to emit the activating light. Said another way, the illustrative two light sources 30, 32 are a single light source (or single set of light sources), which emits at a lower intensity during the guidance mode so as to project the visually perceptible pattern, but with the intensity too low to mark the photochromatic dye. When the “mark” button is pushed in this embodiment, the response is to increase the power of the light source to a higher intensity that is effective (over the exposure time) to mark the dye.

The visible light sources 30 and/or the dye marker light sources 32 can comprise any suitable light source, such as semiconductor lasers (e.g. edge emitting lasers, vertical cavity surface emitting lasers (VCSELs) or so forth), light emitting diodes (LEDs), or so forth, optionally further including optics such as focusing lenses (e.g. a spherical or cylindrical lens focusing the light to a point or line respectively), backing parabolic reflectors focusing the light to a point or line, arrangements of lasers and/or LEDs and/or optics generating more complex patterns such as crosses, and/or so forth). The dye marker light source(s) 32 have a wavelength and intensity that is effective to mark the chosen photochromatic dye. Commonly, the dye marker light source(s) 32 suitably emit in the ultraviolet (UV), since the dye is usually insensitive to visible light (otherwise it would be marked by the ambient room lighting) and the higher photon energy of UV photons is operative to mark the dye. In embodiments employing direct marking, the marker light source(s) should have a wavelength and intensity chosen to penetrate the skin sufficiently to leave visible skin damage, but to not penetrate so deep as to generate deep skin damage that could be very painful and/or harmful to the patient.

During the imaging session, the patient has the dye 34 applied and is marked using the marker light source(s) 32 as described, and the patient undergoes medical imaging to acquire planning images. Thereafter, a dosimetrist, radiologist, oncologist, and/or other medical professional(s) utilize the medical images to construct and optimize the dose plan. At some later time (typically one or more days later), the patient is moved to the RT device 14. The RT device 16 includes the light bridge 15 with one or more second visible light sources 36 disposed in fixed position with respect to the RT device and configured to emit a visible light pattern onto a patient to be treated with the RT device. Prior to radiation delivery, the patient is positioned so that the light emitted from the second visible light sources 36 overlay the marking applied by the imaging light bridge 13 to ensure that the RT is delivered to the correct location on the patient. Notably, the RT light bridge 15 typically does not include any analog to the marker light source(s) 32 of the imaging light bridge 13, because there is no need to mark the patient at the RT device.

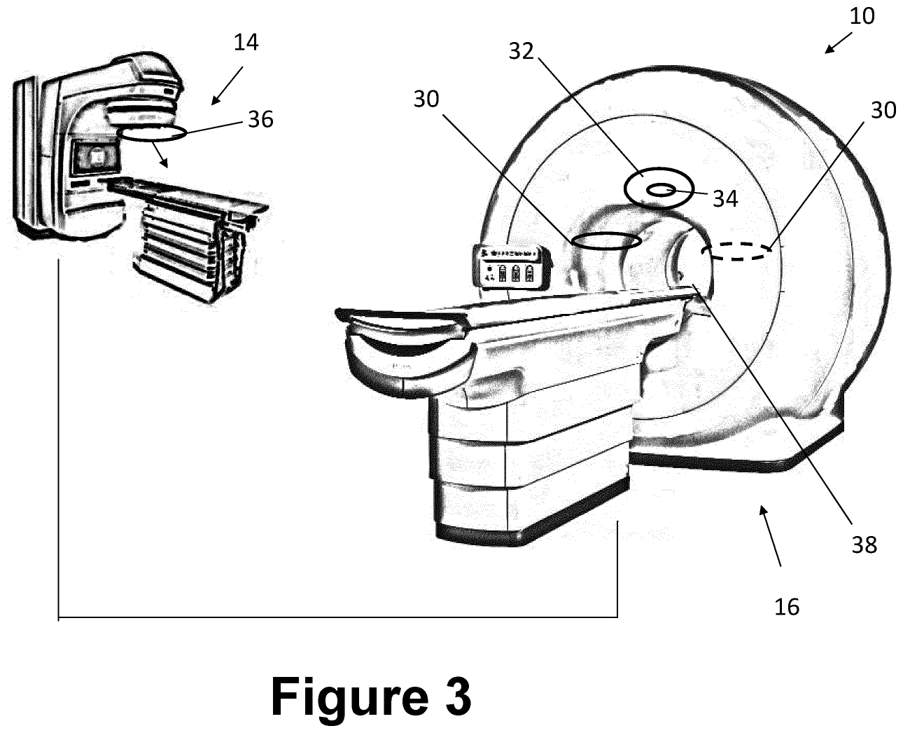

FIG. 3 shows an alternate embodiment of the system 10. In this embodiment, the positioning device 12 is removed, and the visible light sources 30 and the dye marker light sources 32 are disposed in a bore 38 of the medical imaging device 16. As shown in FIG. 3, the visible light sources 30 and the dye marker light sources 32 are disposed around a perimeter of the bore 38 and disposed above the patient as positioned at the entrance to, or within, the bore. In this embodiment (and if the imaging device 16 is an MRI), the dye 34 can optionally include magnetic particles that can be visualized in images of the patient obtained by the medical imaging device 16.

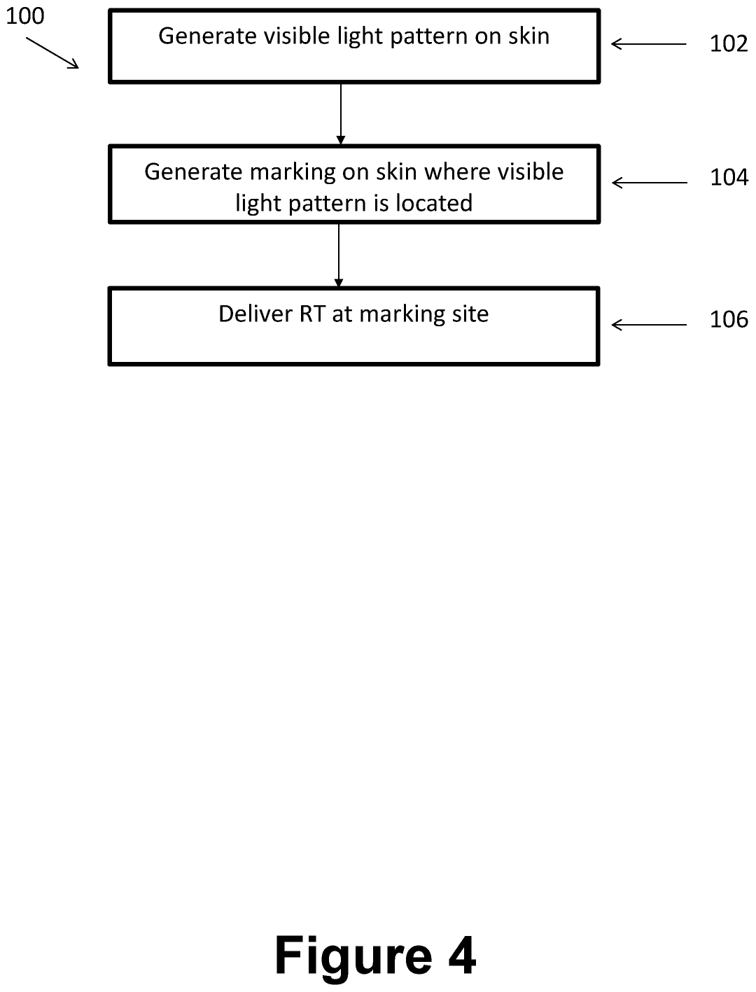

With reference to FIG. 4, an illustrative embodiment of an RT treatment planning method 100 is diagrammatically shown as a flowchart. At an operation 102, light is emitted from the visible light sources 30 towards the patient to generate a visible light pattern on skin of the patient overlaying one or more areas to be treated with RT. At an operation 104, a marking is generated on the skin of the patient where the visible light pattern is located with the photochromatic dye 34 delivered by the dye marker light sources 32. At an operation 106, RT is delivered to the patient with the RT device 14 at the area underlying the marking on the skin of the patient.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A positioning device for use in radiation therapy (RT), the positioning device comprising:
one or more dye marker light sources disposed in fixed position with respect to the medical device and configured to emit activating light onto the patient to be imaged or treated with the medical device which is effective to visually mark an associated photochromic dye disposed on the patient.

2. The positioning device of claim 1, wherein the one or more dye marker light sources comprise one or more ultraviolet (UV) light sources.

3. The positioning device of claim 1, further including:
one or more visible light sources disposed in fixed position with respect to an associated medical device and configured to emit a visible light pattern onto a patient to be imaged or treated with the medical device.

4. The positioning device of claim 3, wherein:
the one or more visible light sources are configured to operate in a guidance mode in which the one or more visible light sources emit the visible light pattern onto the patient; and
the one or more dye marking light sources comprise the one or more visible light sources further configured to operate in a marking mode to emit the activating light which is effective to visually mark the associated photochromic dye disposed on the patient.

5. The positioning device of claim 1, wherein the activating light is not effective to visually mark skin of the patient.

6. The positioning device of claim 1, wherein the one or more dye marker light sources are configured to emit the activating light onto the patient which is effective to visually mark the photochromic dye disposed on the patient by one of:
inducing color and/or opacity in the portion of the photochromic dye disposed on the patient that is illuminated by the activating light; or
inducing a color change in the portion of the photochromic dye disposed on the patient that is illuminated by the activating light.

7. The positioning device of claim 1, wherein the one or more dye marking light sources are configured to emit the activating light onto the patient to visually mark the associated photochromic dye disposed on the patient with one or more lines.

8. The positioning device of claim 7, wherein the one or more dye marker light sources are disposed in the fixed position with respect to the associated medical device such that at least one of the one or more lines is aligned with an anatomical direction of the patient.

9. The positioning device of claim 1, wherein the one or more dye marking light sources are configured to emit the activating light onto the patient which is effective to visually mark the associated photochromic dye disposed on the patient with one or more shapes.

10. The positioning device of claim 9, further comprising: a light bridge fixedly positioned respective to the associated medical device, wherein the one or more dye marker light sources are disposed on the light bridge in the fixed position with respect to the medical device.

11. A medical system, comprising:
a medical imaging device; and
a positioning device as set forth in claim 1, wherein the one or more visible light sources are disposed in fixed position with respect to the medical imaging device and the one or more dye marker light sources are disposed in fixed position with respect to the medical imaging device.

12. The medical system of claim 11, further comprising:
a radiation therapy device; and
one or more second visible light sources disposed in fixed position with respect to the radiation therapy device and configured to emit a visible light pattern onto a patient to be treated with the radiation therapy device.

13. A medical system, comprising:
one or more visible light sources disposed on an outer circumference of a bore of a medical imaging device and configured to emit a visible light pattern comprising one or more lines or shapes onto a patient to be imaged or treated with the medical imaging device; and
one or more marker light sources disposed on an outer circumference of the bore and configured to emit activating light onto the patient to be imaged or treated with the medical imaging device which is effective to visually mark skin of the patient or an associated photochromic dye disposed on skin of the patient with a marking comprising one or more lines or shapes coinciding with the one or more lines or shapes of the visible light pattern.

14. The medical system of claim 13, wherein:
the one or more visible light sources are configured to operate in a guidance mode in which the one or more visible light sources emit the visible light pattern onto the patient; and
the one or more marking light sources comprise the one or more visible light sources further configured to operate in a marking mode to emit the activating light which is effective to visually mark the skin or associated photochromic dye.

15. The medical system of claim 13, wherein the one or more marker light sources are effective to visually mark a napthopyran photochromic dye disposed on skin of the patient.

16. The medical system of claim 13, wherein the one or more marker light sources are configured to emit the activating light onto the patient which is effective to visually mark a photochromic dye disposed on skin of the patient by inducing color and/or opacity in the portion of the photochromic dye that is illuminated by the activating light.

17. The medical system of claim 13, wherein the one or more marker light sources are configured to emit the activating light onto the patient which is effective to visually mark a photochromic dye disposed on skin of the patient by inducing a color change in the portion of the photochromic dye that is illuminated by the activating light.

18. The medical system of claim 13 wherein the one or more marker light sources are configured to emit the activating light onto the patient to visually mark the associated photochromic dye disposed on the patient with one or more lines or shapes.

19. The medical system of claim 13, wherein the activating light is not effective to visually mark skin of the patient.

20. A method of treating a patient with radiation therapy (RT), the method comprising:
emitting light towards the patient to generate a visible light pattern on skin of the patient overlaying one or more areas to be treated with RT;
generating, with a photochromatic dye delivered by one or more dye marker light sources, a marking on the skin of the patient where the visible light pattern is located; and
delivering RT to the patient at the area underlying the marking on the skin of the patient.
